(19) United States
(12) Patent Application Publication
(10) Pub. No.: US 2025/0127535 A1
(43) Pub. Date: Apr. 24, 2025

(54) SYSTEM, METHOD, AND DEVICES FOR TISSUE MANIPULATION USING ELECTRONICALLY STEERABLE ULTRASOUND TRANSDUCER

(71) Applicant: Pulsemedica Corp., Edmonton, Alberta (CA)

(72) Inventors: Nir Katchinskiy, Edmonton, Alberta (CA); Christopher Ceroici, Edmonton, Alberta (CA)

(21) Appl. No.: 18/574,431

(22) PCT Filed: Jun. 29, 2022
(86) PCT No.: PCT/CA2022/051038

§ 371 Date: Dec. 27, 2023

Related U.S. Application Data
(60) Provisional application No. 63/216,637, filed on Jun. 30, 2021.

(51) Int. Cl. A61B 17/32; A61B 8/00; A61F 9/007

(52) U.S. Cl. CPC A61B 17/320068; A61B 8/5261; A61F 9/007; A61B 2017/320069; A61B 2017/320082

(57) ABSTRACT

An ultrasonic transducer may be arranged within an alignment housing that can be secured to a surface of a patient. The alignment housing may include one or more ports that allow an acoustic coupling medium to be injected into a space between the surface the device is secured to and the transducer array.

### RELATED APPLICATIONS

The current application claims priority to previously filed U.S. Provisional Patent Application No. 63/216,637 Filed Jun. 30, 2021 and titled “SYSTEM, METHOD, AND DEVICE FOR TISSUE CUTTING, ABLATION, OR MANIPULATION USING ELECTRONICALLY STEERABLE ULTRASOUND TRANSDUCER” and PCT application PCT/CA2022/050582 Filed Apr. 14, 2022 and titled “BIO-MEDICAL IMAGING DEVICES, SYSTEMS and METHODS OF USE”, both of which are incorporated herein by reference in their entirety.

### TECHNICAL FIELD

The current application relates to electronically steerable ultrasonic transducers, and in particular to steerable ultrasonic transducers for cutting, ablating or manipulation of tissue.

### BACKGROUND

There are numerous conditions that require surgical removal of internal tissue such as cancer or non-cancerous tumors. Some techniques for removal of the affected tissue includes surgery, radiation, chemotherapy and laser treatment. One particular non-invasive technology for treatment of internal body tissue is called high-intensity focused ultrasound (HIFU). With HIFU, high intensity ultrasound energy is focused at a desired treatment volume. The energy causes tissue destruction via both thermal and mechanical mechanisms.

One of the drawbacks of using HIFU to treat internal body tissues is the relatively large focal spot size that could result in damage to surrounding healthy tissue, as well as precisely controlling the location of the focal spot. Given these problems, there is a need for a method of treating internal body tissues accurately.

### SUMMARY

In accordance with the present disclosure there is provided an ultrasound transducer device comprising: an alignment body defining an interior chamber, the alignment body configured to be physically coupled to a surface of a body portion to be treated; an ultrasound transducer array sized to be received within the interior chamber of the alignment body, the ultrasound transducer array comprising a plurality of individual ultrasound transducers; and an inlet port for injecting an acoustic coupling medium into the interior chamber between the ultrasound transducer array and the surface the portion of the body to be treated when the alignment body is physically coupled to the surface of the body portion to be treated.

In a further embodiment of the ultrasound transducer device, the ultrasound transducer device further comprises: a sealing mechanism adapted to seal the ultrasound transducer device to a surface of a portion of a body to be treated when in use.

In a further embodiment of the ultrasound transducer device, the sealing mechanism comprises a suction chamber surrounding at least a portion of the interior chamber.

In a further embodiment of the ultrasound transducer device, the suction chamber is formed as part of the alignment body defining the interior chamber.

In a further embodiment of the ultrasound transducer device, the suction chamber comprises a contacting ring surrounding the interior chamber that contacts the surface of the body portion and seals the alignment body to the surface when suction is applied to the suction chamber.

In a further embodiment of the ultrasound transducer device, the sealing mechanism comprises an adhesive surface to which an adhesive can be applied and pressed against the portion of the body to be treated and seal the ultrasound transducer device to the surface of the portion of the body.

In a further embodiment of the ultrasound transducer device, the ultrasound transducer device further comprises: an outlet port allowing a gas to be evacuated from the interior chamber.

In a further embodiment of the ultrasound transducer device, the outlet port further allows a gas to enter the interior chamber.

In a further embodiment of the ultrasound transducer device, the inlet port further allows the acoustic coupling medium to be extracted from the interior chamber.

In a further embodiment of the ultrasound transducer device, the ultrasound transducer device further comprises: an extraction port allowing the acoustic coupling medium to be extracted from the interior chamber.

In a further embodiment of the ultrasound transducer device, the plurality of individual ultrasound transducers are individually adjustable to allow the focusing of the ultrasound beam to electronically steered.

In a further embodiment of the ultrasound transducer device, the plurality of individual ultrasound transducers of the ultrasound transducer array are arranged in a spherical shape.

In a further embodiment of the ultrasound transducer device, the ultrasound transducer array comprises a portion providing an optical lens.

In a further embodiment of the ultrasound transducer device, the transducer array comprises an optical path for coupling the optical lens to at least one of an optical imaging system and an optical treatment system.

In a further embodiment of the ultrasound transducer device, the ultrasound transducer array comprises one or more registration marks allowing a positioning of the ultrasound transducer array to be determined.

In a further embodiment of the ultrasound transducer device, the ultrasound transducer array is secured in a fixed position within the interior chamber.

In a further embodiment of the ultrasound transducer device, the ultrasound transducer array is moveable within the interior chamber.

In a further embodiment of the ultrasound transducer device, the ultrasound transducer device further comprises: one or more actuators for moving the transducer array within the interior chamber.

In a further embodiment of the ultrasound transducer device, the one or more actuators comprise one or more piezo-electric actuators.

In a further embodiment of the ultrasound transducer device, the one or more actuators are arranged to move the transducer array closer to or away from the surface the portion of the body to be treated.

In a further embodiment of the ultrasound transducer device, an aperture of the ultrasound transducer array is sized approximately to a size of an eye.

In accordance with the present disclosure there is further provided an ultrasound treatment system comprising: an ultrasound transducer device according to any of the embodiments described above; and a computing device coupled to the ultrasound transducer array and configured to provide a driving signal to the ultrasound transducer array.

In a further embodiment of the ultrasound transducer system, the ultrasound transducer system further comprises a vacuum pump connected to the suction chamber.

In a further embodiment of the ultrasound transducer system, the driving signal to the ultrasound transducer array provides high-intensity focused ultrasound (HIFU) treatment.

In a further embodiment of the ultrasound transducer system, the ultrasound transducer system further comprises an optical coherent tomography (OCT) imaging system.

In accordance with the present disclosure there is further provided a method of performing an ultrasonic procedure comprising: affix an alignment housing to a patient at a treatment location; couple an ultrasonic transducer to the alignment housing; add an acoustic coupling media to the alignment housing between the ultrasonic transducer and the patient; and performing an ultrasonic procedure on the treatment location.

In a further embodiment of the method, affixing the alignment housing comprises applying a suction to the treatment location through the alignment housing.

In a further embodiment of the method, affixing the alignment housing comprises applying an adhesive to a portion of the alignment housing that will be in contact with the treatment location.

In a further embodiment of the method, the ultrasonic transducer comprises an optical pathway for coupling to an optical imaging system, and the method further comprising: generating an image of at least a portion of the treatment location using the optical imaging system; identifying a target location in the image; and performing the ultrasonic procedure on the target location.

In a further embodiment of the method, the method further comprises: generating an ultrasound image of at least an overlapping portion of the treatment location using ultrasound; and aligning the ultrasound transducer and the optical imaging system using the image and the ultrasound image.

### BRIEF DESCRIPTION OF THE DRAWINGS

Further features and advantages of the present disclosure will become apparent from the following detailed description, taken in combination with the appended drawings, in which:

FIG. 1 depicts ultrasonic treatment of an eye;

FIG. 2 depicts pressure amplitudes during ultrasonic treatment of an eye;

FIG. 3 depicts an ultrasound transducer device;

FIG. 4 depicts a further ultrasound transducer device;

FIG. 5 depicts an ultrasound treatment system;

FIG. 6 depicts a further ultrasound transducer device suitable for internal use;

FIG. 7 depicts components of an imaging system incorporating an ultrasonic transducer device;

FIG. 8 depicts a method of performing a procedure using an ultrasonic device; and

FIG. 9 depicts a method of performing a procedure using an imaging system and an ultrasonic device.

### DETAILED DESCRIPTION

An ultrasonic treatment device may have a transducer array within an alignment housing. The alignment housing can be secured to a location being treated. Securing the alignment housing to the treatment location aligns the transducer array to the treatment location allowing for the precise targeting of the ultrasonic waves. The device may also include one or more ports that can be used to inject, and or remove, an ultrasonic coupling medium into the device in order to fill a void between the surface the device is secured to and the transducer array.

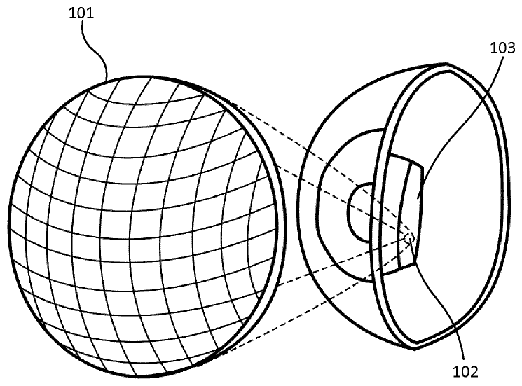

FIG. 1 depicts ultrasonic treatment of an eye. The figure shows a high intensity ultrasonic transducer array 101, which may be a 2-dimensional transducer array, with an ultrasonic focus that is electronically controlled towards a particular treatment location 102 for the purpose of manipulating tissue at or within the target location, depicted as the crystalline lens 103. The tissue manipulation may include for example an incision of the tissue, emulsification of tissue, bubble formation within the tissue, ionization of the tissue, removal of the tissue, vaporization of the tissue, softening or liquefaction of the tissue, as well as controlling or affecting the motion of tissue or components of tissues. These are only examples of possible tissue manipulations and other tissue manipulations may be performed. The particular treatment location and size may be dependent upon the size of the ultrasonic transducer. The figure depicts an example where precise incisions can be made inside the crystalline lens 103 of the eye, however the precise incision can target other treatment locations including in the vitreous humor, anterior chamber of the eye as well as other structures such as the cornea and lens capsule. Further, although depicted as being used for incisions or treatments of the eye, it is possible to apply a similar ultrasonic device for treatment of other locations including internal locations and external locations.

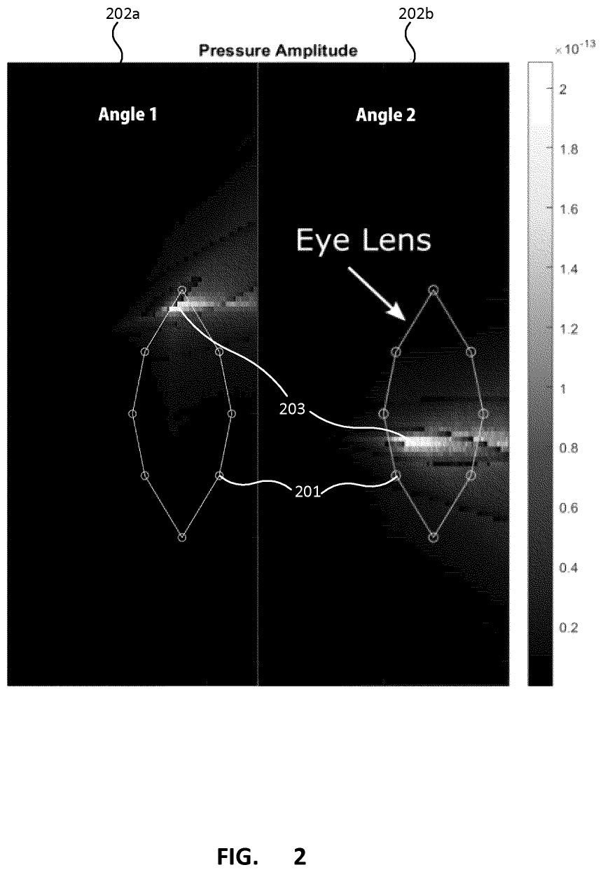

FIG. 2 depicts pressure amplitudes during ultrasonic treatment of an eye. The figure shows an example of pressure amplitude that is generated by the system inside the crystalline lens 201 from two different angles 202a, 202b. It can be seen that the focal spot of the pressure wave, 203, is generated inside the lens to perform precise incision (emulsification, bubbles, ionization, removal, vaporization).

Although described as being used for incisions, it is possible to use the ultrasonic transducer for other purposes, including for example ultrasound imaging, as well as possible tissue manipulation in addition to, or as an alternative to incisions. For example, the ultrasonic waves from the ultrasonic transducer may be used to control or affect the motion of tissue or components of tissues.

Focusing of the ultrasonic waves at a particular location in may require the ultrasonic transducer be held in a fixed position relative to the particular location. Securing the ultrasonic transducer to a location may be possible for certain locations on a patient or target. However for other locations, including for example a patient's eye, securing the ultrasonic transducer in position can be difficult. As described further below, an alignment housing can be provided that can be secured to the patient to provide a mounting location for securing the ultrasonic transducer.

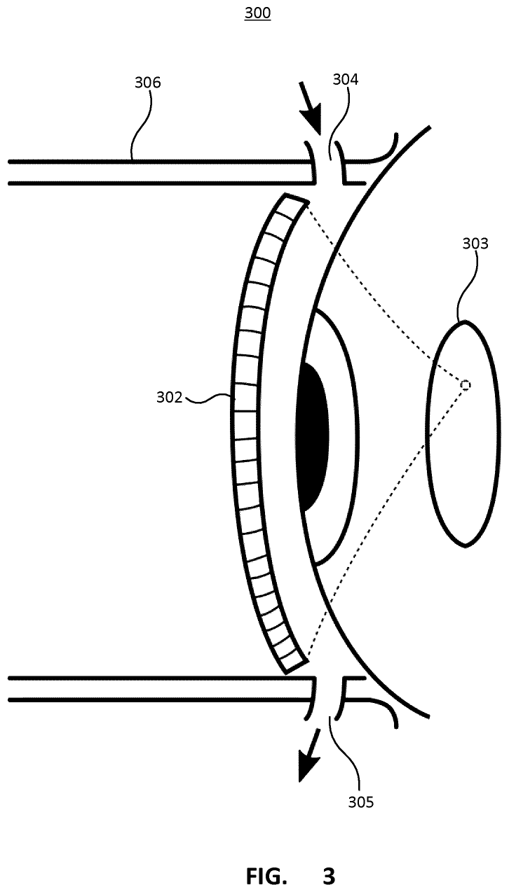

FIG. 3 depicts an ultrasound transducer device. The figure depicts a device 300 for performing precise incisions inside a crystalline lens inside an eye. The device comprises a transducer array 302 that can be coupled to an alignment housing 306. An eye, with crystalline lens 303, is in contact with the device's alignment housing 306. The alignment is constructed such that an ultrasonic coupling medium can be injected into a volume that receives an ultrasonic transducer array. The volume may be defined by a wall or walls that include one or more ports 304, 305. The coupling medium may be injected through a port 305, while excess air/liquid can be expelled or removed through a second port 304. The housing may have a surface that can be placed on the patient's eye similar to a contact lens. The surface may be a ring portion of a sphere or similar surface. The contact surface of the housing may be secured to the patient's eye by way of surface tension or possible with a vacuum or suction to secure the housing in place relative to the patient's eye. The housing provides an alignment body that defines an interior chamber adjacent to treatment area such as the eye. A 2D array ultrasonic transducer, 301, that has electronically steerable focus point 302 can be received within a portion of the housing. The housing aligns the transducer relative to the eye. The ultrasonic coupling medium can be inserted into the space of the housing between the 2D array ultrasonic transducer and the eye. Although described as a 2D array, it is possible the ultrasonic transducer may have a 3D array.

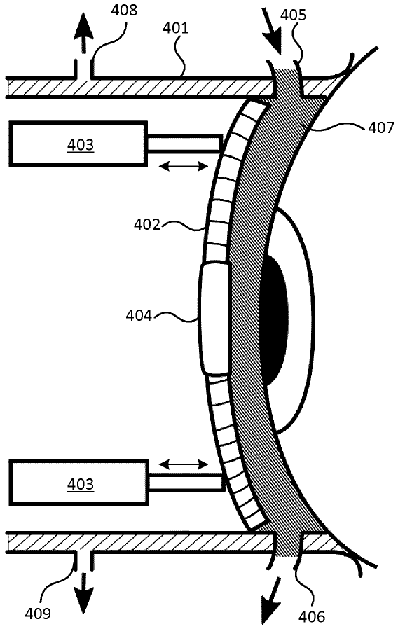

FIG. 4 depicts a further ultrasound transducer device. The figure depicts the device that is in contact with an eye, where the housing of the device 401 is touching the surface of the eye. The device is similar to the device described above with regard to FIG. 3 and comprises a 2D electronically steerable ultrasound transducer 402. However the device of FIG. 4 further comprises one or more (two are depicted in FIG. 4) actuators 403, which may be for example piezoelectric actuators, that can be used to position the transducer within the housing. For example, the actuators may move the transducer further or closer to the eye. The actuators may be provided by various actuator devices including, for a piezoelectric actuator. As the transducer is moved further/closer to the eye, the coupling media may be added or removed through the ports. The actuators 403 may be individually controlled in order to adjust the orientation of the transducer and possibly adjust the natural focus of the array. Additionally, the ultrasound transducer in FIG. 4 is depicted as having an optical window 404 for coupling an imaging system such as, but not limited to, Optical Coherence Tomography, Scanning Laser Ophthalmoscope, optical microscope. Further, although not depicted in the figure, it is possible to include one or more ultrasound sensors which may be used to perform ultrasonic imaging.

Similar to the device described above with reference to FIG. 3, the device of FIG. 4 may have inlet, 405, and outlet, 406, ports for ultrasonic coupling medium, 407, that can be injected into the void between the eye and the transducer. Although depicted as having separate inlet and outlet ports, it is possible for a single port to be used for both injecting and removing the ultrasonic coupling medium. Although described as an ultrasonic coupling medium, the fluid added/removed through the ports 405, 406 may be various different types of media that may have different properties or characteristics. Further, the fluid present between the transducer array 402 and the treatment location, depicted as the eye in FIG. 4, may be varied during the procedure. For example, during an imaging portion of a procedure which may use optical imaging devices arranged to image the treatment location through the optical window 404, the fluid present may be selected to have particularly desirable optical properties for the imaging system. During an ultrasound treatment portion of the procedure, the fluid previously used for its optical properties may be removed and replaced with an ultrasound coupling media having properties that are desirable for the ultrasound procedure. Additionally, the fluid may be used for other purposes. For example, the flow may flow through the device during the procedure possibly to remove debris or contaminants from the treatment location, deliver medication or other compounds to the treatment location, etc. Further, the fluid may be heated or cooled in order to possibly heat/cool the treatment location as well as the ultrasound array.

The system's housing can be constructed such that its perimeter can be connected or coupled securely to the surface of the eye. For example, the housing may have a perimeter that can contact the eye and be secured using suction that is generated by suction outlets 408, 409. The perimeter may provide a suction or vacuum chamber that is open on the bottom where the perimeter contacts the patient's eye. The suction outlets 408,409 can create a vacuum or suction that secures the perimeter to the eye. Alternatively, the vacuum chamber may have a deformable bottom surface that contacts the patient's eye and deforms under suction to create a suction on the patient's eye.

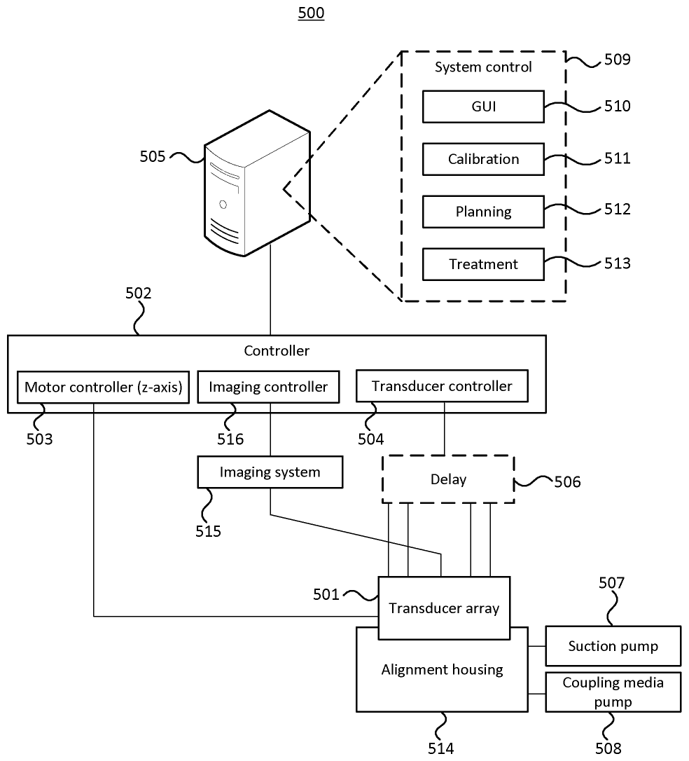

FIG. 5 depicts an ultrasound treatment system. This figure depicts an entire system for the device. A personal computer (PC) or similar computing device may be provided to control the treatment system. The PC 505 may communicate with one or more controllers. For example, a device controller 502 may control operation of the device. The device controller may communicate with one or more motor controllers 503, that control the mechanical (e.g. piezoelectric) actuators for positioning the transducer. Additionally or alternatively, the device controller may communicate with a transducer controller 504. The transducer controller communicates with the transducer array, 501 through a series of delay lines 506 which may be used to steer the ultrasonic wave generated from the transducer array. The controller may also communicate with a suction pump 507 and the acoustic matching pump 508. The suction pump may apply suction to the alignment housing in order to secure the device to the patient's eye, while the acoustic matching pump may control the acoustic matching fluid pumped into and/or out of the device from between the eye and the transducer. The PC has means of controlling the system and display system parameters using a system control module 509 that consists of GUI 510. The GUI provides access to one or more modules including for example a calibration module 511, a planning module 512, and a treatment module 513. The calibration module 511 can be used to calibrate one or more parameters of the system. The planning module 512 may provide an interface for planning one or more treatment procedures using the system and the treatment module 513 may provide an interface for carrying out one or more treatment procedures using the system.

The above has described various devices and systems for treating an eye condition using ultrasound. A similar device may be used for ultrasonic treatment as well as ultrasonic imaging or other tissue manipulation, of other portions of the body such as the skin, or possibly internally. The alignment housing 514 may be affixed to the treatment location, for example using surface tension, suction, vacuum or other possible methods including possibly adhesives or mechanical means such as straps or tape. The alignment housing may be removed from the patient after the treatment or procedure. Alternatively, the alignment housing may be left in place for subsequent treatment or procedures. Leaving the alignment housing in place may allow the ultrasonic transducer to be re-attached at the same location.

The alignment housing 514, and possibly the transducer array 501, may comprise one or more optical components such as lenses, fibers, and/or channels for optically coupling to an imaging system 515 that may be controlled by an imaging controller 516. The imaging system may comprise a plurality of different imaging modalities such as an SLO imaging system an OCT imaging system, a video imaging system, etc. The different imaging modalities may be controlled in order to be co-aligned with each other. The imaging systems may generate images using one or more light sources including both coherent light sources such as continuous wave lasers and/or pulsed lasers as well as non-coherent light sources. The alignment housing, and/or the transducer array, may have one or more controllable optical components in order to control the alignment of the imaging system 515 relative to the ultrasound transducer array. Additionally or alternatively, the transducer array may have one or more controllable components for controlling the alignment of the ultrasound transducer array relative to the optical imaging system 515.

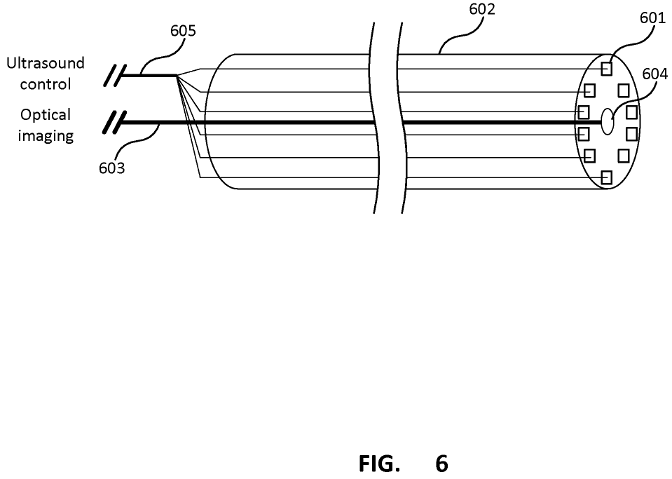

FIG. 6 depicts a further ultrasound transducer device. The transducer device may be used with an alignment housing and may be suitable for internal and/or external use. The system comprises a 2D electronically steerable ultrasound array 601 arranged at the end of a flexible scope 602. In the core of the scope, there can be an optical fiber 603, which may terminate at an optical window or lens 604 that provides access to optical imaging. The optical fiber may be controllably moveable, for example using actuators, within the scope in order to control the alignment of the optical imaging system with the ultrasound transducer. Additionally or alternatively, the scope 602 may include one or more optical components such as lenses for adjusting the focus of the imaging system. The transducer array 601 is connected to the system using wires 606, that are connected to the ultrasound transceivers. The ultrasonic array may be positioned adjacent a treatment area using the optical imaging. The optical imaging system may provide one or more imaging modalities that can be aligned with each other and may be used to identify the treatment location. The ultrasound transducer array 601 may include a sensor (not depicted) in order to generate ultrasound image(s) of the treatment location. The ultrasound image(s) may be aligned with the optical images by identifying the same features within the images. The scope may also include one or more tubes, or channels (not shown in FIG. 6) arranged within the scope for an ultrasonic coupling medium and for providing suction which may be used to secure the device in a desired location. The scope may act as the alignment housing described above or the scope may be connected to a separate alignment housing secured at or near the treatment location.

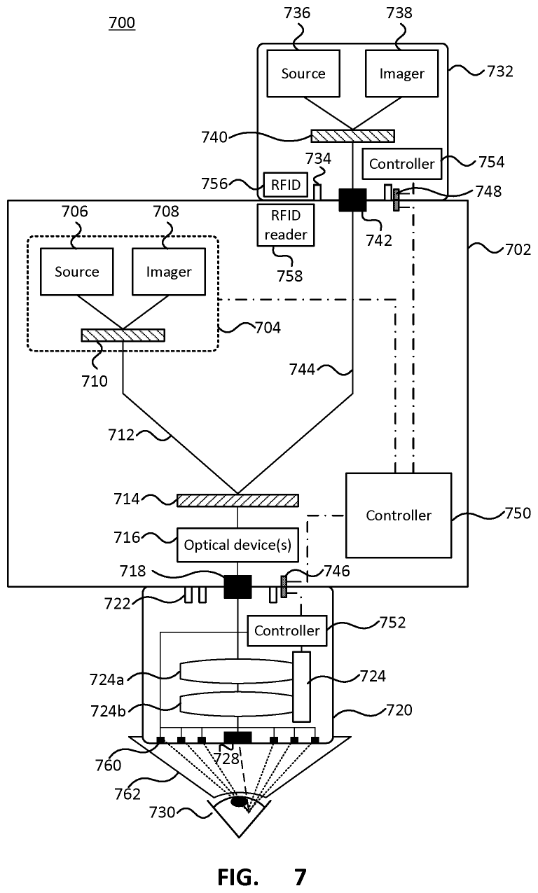

FIG. 7 depicts components of an imaging system incorporating an ultrasonic transducer device. The system 700 is similar to that described in PCT application PCT/CA2022/050582, filed Apr. 14, 2022 and incorporated herein by reference in its entirety. The system 700 comprises a base device 702 that has input and output modules physically and optically coupled to it. The base device 702 provides imaging functionality 704 with an imaging source 706 and imaging sensor 708. The imaging functionality 704 may incorporate additional optics and/or components not depicted in FIG. 2. The imaging source and sensor may vary depending upon the imaging functionality provided. For example, the imaging functionality may provide a digital ophthalmoscope using a white light source and a 2D image sensor, such as a CMOS sensor along with appropriate optics. Alternatively, the imaging functionality may provide SLO (Scanning Laser Ophthalmoscopy) functionality that uses a laser source with scanning optics such as galvanometers for scanning the laser across the patient's eye to generate an image of the eye.

The base device 702 has an optical path 712 that provides an optical path between the imaging functionality 704 and a beam combiner/splitter device 714 that is optically coupled to one or more optical devices 716 such as focusing optics. The focusing optics may comprise one or more adjustable lenses. Additionally or alternatively, the optical devices may comprise scanning optics for controlling a direction of the light on the patient's eye. The base device 702 comprises an optical output port 718 that allows the optical devices to be optically coupled to a corresponding optical coupler of an output optical module 720 that is mechanically coupled to the base device by a physical interface connector 722. The physical connection may comprise cooperating features on both the base device and the output module 720.

The output module 720 is depicted as providing optics 724 that comprise moveable lenses 724a, 724b that can be used to adjust a focusing depth of the light source and imager through an output port 728 onto a patient's eye 730. Additionally the output module 720 includes an ultrasonic transducer array 760 that is electronically steerable onto a particular location within the patient's eye and arranged at an end of the output module 720. The optical imaging systems may be targeted at the same location as the ultrasound, or may be targeted at a different location. The output module 720 may be physically coupled to an alignment housing 762 that in turn is physically coupled to the patient's eye. The alignment housing comprises one or more ports for adding or removing an acoustic coupling media between the transducer and the patient's eye.

Combining one or more optical imaging modalities together along with an ultrasonic transducer that can be aligned with the optical imaging systems can be used to target specific locations which may be precisely identified with one or more of the optical imaging systems with ultrasound treatment. Further, the transducer may be provided with one or more ultrasound sensors that can be used to generate images from the ultrasound. Further, the combination of the imaging systems, or the light sources of one or more of the imaging systems, and the ultrasonic sensor may be used to provide photoacoustic imaging capabilities to the system. The optical imaging systems and the ultrasound transducer and sensors may be used to generate images of the same features within the treatment location. By identifying the same physical features within the different images, it is possible to align the different imaging systems so that the same locations can be targeted using the different systems. For example, with the optical imaging systems aligned with the ultrasound imaging system, it is possible to identify treatment targets with the optical imaging system and then treat them using the ultrasonic transducer of the ultrasound imaging system. Additionally or alternatively, one or more lasers, which may be provided as a light source of an imaging system or as a separate treatment laser, may be used for treatment either on its own or in combination with ultrasound.

In addition to the output module 720, the ophthalmological system 700 may comprise an input optical module 732 that is mechanically coupled to the base device 702 using a physical interface 734 between the module and the device. The physical interface 734 may comprise cooperating features on each of the input module 732 and base device 702 that align a lock the input module in place relative to the base device. As depicted, the input module 732 provides a further light source 736 and corresponding imaging sensor 738 along with a beam combiner/splitter or other optical devices 740 for directing the light from the source to an optical interface 742 between the input module 732 and the base device 702 and from the optical interface 742 to the sensor 738. The optical interface 742 couples the input module to a second optical path 744 of the base device 702 that in turn is optically coupled to the output module 720 through the beam combiner/splitter 714 and optical devices 716.

The input module may provide a different imaging modality from the imaging functionality 704 of the base device. For example, the base device may be provided with SLO imaging functionality while the input module 732 may provide OCT (Optical Coherence Tomography) imaging. It will be appreciated that not all of the imaging components for providing OCT imaging are depicted in FIG. 7.

The base device 702 may further include a controller 750 for controlling operation of the ophthalmological system 700. The controller may control operating of the imaging functionality of the base device as well as providing a user interface for operating the system 700. The user interface may be provided as a graphical user interface displayed on a display (not shown) of the base device or may comprise an interface provided on a remote device that may be in communication with the controller via a communication module such as a Wi-Fi® radio, NIC (Network Interface Card), serial communication interfaces or other communication devices such as Bluetooth radios. The controller 750 may be provided as one or more individual control devices such as FPGAs (Field Programmable Gate Arrays), ASICs (Application Specific Integrated Circuits), and/or Central Processing Units (CPUs) that are operating together to provide the functionality of the system 700.

The base device 702 described above provide internal imaging functionality as well as interfaces for coupling, both mechanically and optically, input and output modules to the base device. In addition to the physical and optical interfaces between the input/output modules and the base device, an electrical interface 752 for the output module and an electrical interface 754 for the input module. The electrical interfaces 752, 754 provide one or more electrical connections between the modules and base device. The electrical interfaces may couple the input/output modules to the controller 750 of the base device in order to provide electrical power to components of the input/output modules as well as provide an electrical communication path. As depicted the input and output modules may each include one or more controllers 752, 754. The controllers of the modules may communicate with the controller of the base device in order to co-ordinate operation of the modules. Additionally, the controllers of the input and output modules may provide information useful for configuring the modules with the base device. For example, the controller 752 of the output module may communicate identifying information stored in the module to the controller of the base device 702. The identifying information may provide information to the base device on how the base device can control the output module as well as other information such as possibly other requirements and/or limitations of the output module.

As described above, the controller of an output or input module may communicate identifying information about the module. The identifying information may be provided in various ways such as using an RFID tag 756 on the module and an RFID tag reader 758 on the base device 702 that can communicate the module's identifying information stored in the RFID tag. In addition to providing identifying information to the base device, the base device may also provide identifying information of the base device to the modules to allow the modules to identify the base device and possibly adjust the operation of the input or output module to operate with the base device.

Using a base ophthalmological device as described above, it is possible to provide additional modules, whether input and/or output modules that can extend the functionality of the base device. Different input and output modules may be combined in different kits to provide various functionality.

The transducer array described above is depicted as having the transducer elements and the optical window arranged symmetrical about a longitudinal axis of the transducer. However, it is possible for the optical elements and/or the transducer elements to be arranged in other configurations.

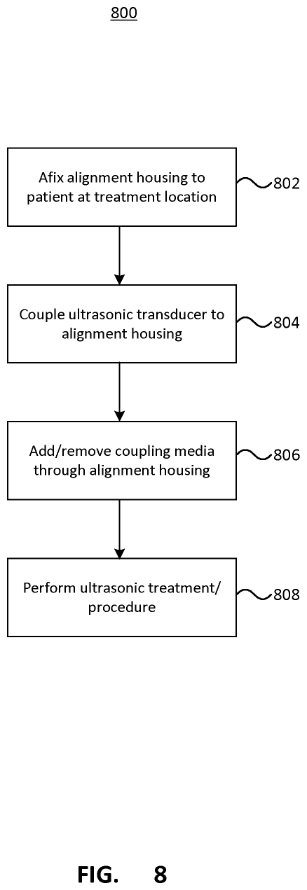

FIG. 8 depicts a method of performing a procedure using an ultrasonic device. The method 800 includes affixing the alignment housing to the patient at the treatment location (802). As described above, the alignment housing may have a surface that is designed to contact the patient at the treatment location. For example, the treatment location may be a patient's eye and the alignment housing may have a surface similar to a contact lens that contacts the patient's eye. An ultrasonic transducer is coupled to the alignment housing (804). The alignment housing and transducer have a corresponding shape and size that allow the transducer to be connected to the alignment housing. Additionally, the alignment housing and transducer may have one or more alignment features that allow the transducer to be connected to the alignment housing at a fixed or known position. Acoustic coupling media may be added and or removed from the alignment housing through one or more ports of the alignment housing (806). The acoustic coupling media improves the acoustic coupling between the transducer and the treatment location. With the acoustic coupling media between the transducer and the treatment location, the ultrasonic treatment or procedure may be performed, such as performing an incision, ablation, or other tissue manipulation. Although the above has described a particular sequence of performing the procedure, namely affixing the alignment housing, coupling the transducer and then adding coupling media, it is possible to perform the steps in different orders. For example, the transducer may first be coupled to the alignment housing which may then be affixed to the patient.

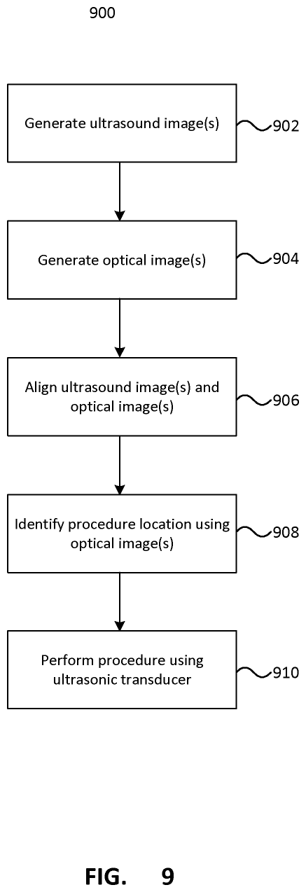

FIG. 9 depicts a method of performing a procedure using an imaging system and an ultrasonic device. The method 900 may be performed by a system that includes both an ultrasonic imaging system comprising an ultrasound transducer and sensor as well as one or more imaging systems comprising a light source and sensor. Ultrasound images are generated of the treatment location, or portion of the treatment location, (902) and optical images are generated of at least a partially overlapping portion of the treatment location (904). The ultrasound and optical images are aligned with each other so that locations in one imaging systems can be mapped to corresponding locations in the other imaging systems. The alignment may be performed by identifying features within the different images, identify corresponding features across the images of different modalities and determining the alignment between the systems based on locations of the corresponding features. The co- alignment of the different imaging systems may be done in software or may be done by physically adjusting relative positioning or focusing of the imaging systems relative to each other.

Once the ultrasound and optical imaging systems are co-aligned, a target location for the procedure may be identified within the optical image(s), or possibly the ultrasound images, or a combination of the optical images and ultrasound images (908). Once the treatment location is identified, the procedure may be performed using the ultrasonic transducer (910). The procedure may also be performed using one or more light sources of the imaging systems or separate treatment lasers.

It will be appreciated by one of ordinary skill in the art that the system and components shown in FIGS. 1-9 may include components not shown in the drawings. For simplicity and clarity of the illustration, elements in the figures are not necessarily to scale, are only schematic and are non-limiting of the elements structures. It will be apparent to persons skilled in the art that a number of variations and modifications can be made without departing from the scope of the invention as defined in the claims.

Numerous additional variations on the methods and apparatus of the various embodiments described above will be apparent to those skilled in the art in view of the above description. Such variations are to be considered within the scope.

1. An ultrasound transducer device comprising:
an alignment body defining an interior chamber, the alignment body configured to be physically coupled to a surface of a body portion to be treated;
an ultrasound transducer array sized to be received within the interior chamber of the alignment body, the ultrasound transducer array comprising a plurality of individual ultrasound transducers; and
an inlet port for injecting an acoustic coupling medium into the interior chamber between the ultrasound transducer array and the surface the portion of the body to be treated when the alignment body is physically coupled to the surface of the body portion to be treated.

2. The ultrasound transducer device of claim 1, further comprising:
a sealing mechanism adapted to seal the ultrasound transducer device to a surface of a portion of a body to be treated when in use.

3. The ultrasound transducer device of claim 2, wherein the sealing mechanism comprises a suction chamber surrounding at least a portion of the interior chamber.

4. The ultrasound transducer device of claim 3, wherein the suction chamber is formed as part of the alignment body defining the interior chamber.

5. The ultrasound transducer device of claim 4, wherein the suction chamber comprises a contacting ring surrounding the interior chamber that contacts the surface of the body portion and seals the alignment body to the surface when suction is applied to the suction chamber.

6. The ultrasound transducer device of claim 2, wherein the sealing mechanism comprises an adhesive surface to which an adhesive can be applied and pressed against the portion of the body to be treated and seal the ultrasound transducer device to the surface of the portion of the body.

7. The ultrasound transducer device of claim 1, further comprising an outlet port allowing a gas to be evacuated from the interior chamber.

8. (canceled)

9. The ultrasound transducer device of claim 1, wherein the inlet port further allows the acoustic coupling medium to be extracted from the interior chamber.

10. The ultrasound transducer device of claim 1, further comprising an extraction port allowing the acoustic coupling medium to be extracted from the interior chamber.

11. The ultrasound transducer device of claim 1, wherein the plurality of individual ultrasound transducers are individually adjustable to allow the focusing of the ultrasound beam to electronically steered.

12. The ultrasound transducer device of claim 1, wherein the plurality of individual ultrasound transducers of the ultrasound transducer array are arranged in a spherical shape.

13. The ultrasound transducer device of claim 1, wherein the ultrasound transducer array comprises a portion providing an optical lens.

14. The ultrasound transducer device of claim 13, wherein the transducer array comprises an optical path for coupling the optical lens to at least one of an optical imaging system and an optical treatment system.

15. The ultrasound transducer device of claim 1, wherein the ultrasound transducer array comprises one or more registration marks allowing a positioning of the ultrasound transducer array to be determined.

16. The ultrasound transducer device of claim 1, wherein the ultrasound transducer array is secured in a fixed position within the interior chamber.

17. The ultrasound transducer device of claim 1, wherein the ultrasound transducer array is moveable within the interior chamber.

18. The ultrasound transducer device of claim 17, further comprising one or more actuators for moving the transducer array within the interior chamber.

19. (canceled)

20. (canceled)

21. (canceled)

22. An ultrasound treatment system comprising:
an ultrasound transducer device according to claim 1; and
a computing device coupled to the ultrasound transducer array and configured to provide a driving signal to the ultrasound transducer array.

23. (canceled)

24. (canceled)

25. (canceled)

26. A method of performing an ultrasonic procedure using an ultrasound transducer device according to claim 1, the method comprising:
affixing an alignment housing to a patient at a treatment location;
coupling an ultrasonic transducer to the alignment housing;
adding an acoustic coupling media to the alignment housing between the ultrasonic transducer and the patient; and
performing an ultrasonic procedure on the treatment location.

27. (canceled)

28. (canceled)

29. The method of claim 26, wherein the ultrasonic transducer comprises an optical pathway for coupling to an optical imaging system, the method further comprising:
generating an image of at least a portion of the treatment location using the optical imaging system;
identifying a target location in the image; and
performing the ultrasonic procedure on the target location.

30. The method of claim 29, further comprising:
generating an ultrasound image of at least an overlapping portion of the treatment location using ultrasound; and
aligning the ultrasound transducer and the optical imaging system using the image and the ultrasound image.
